(19) United States
(12) Patent Application Publication
(10) Pub. No.: US 2008/0221603 A1
(43) Pub. Date: Sep. 11, 2008

(54) ULTRASONIC TREATMENT DEVICE

(76) Inventor: Norihiro Yamada, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/109,920

(22) Filed: Apr. 25, 2008

Related U.S. Application Data
(63) Continuation of application No. PCT/JP2006/321417, filed on Oct. 26, 2006.

(30) Foreign Application Priority Data
Oct. 28, 2005 (JP) 2005-314323

(51) Int. Cl. A61B 17/32

(52) U.S. Cl. 606/169; 601/2

(57) ABSTRACT

An ultrasonic treatment device, includes at least a pair of electrodes disposed so as to be spaced from each other in an insulated state, an actuator main body formed from an electrostrictive polymer that disposed between the respective electrodes, where the electrostrictive polymer is driven in a stretching and shrinking manner when voltage is applied between the respective electrodes, a power source supplying voltage between the respective electrodes, and a treatment portion configured to treat a site to be treated by ultrasonic vibration thereof in linkage with driving of the electrostrictive polymer in the stretching and shrinking manner.

### CROSS REFERENCE TO RELATED APPLICATIONS

This is a Continuation application of PCT Application No. PCT/JP2006/321417, filed Oct. 26, 2006, which was published under PCT Article 21(2) in Japanese.

This application is based upon and claims the benefit of priority from prior Japanese Patent Application No. 2005-314323, filed Oct. 28, 2005, the entire contents of which are incorporated herein by reference.

### BACKGROUND OF THE INVENTION

1. Field of the Invention

The present invention relates to an ultrasonic treatment device used for clotting and incising of a body tissue in such an operation as, for example, a surgical operation.

2. Description of the Related Art

When an abdomen of a patient is opened and is treated with a surgical operation or when a surgical operation is applied under an endoscope, an ultrasonic treatment device is generally used as means for conducting clotting and incising of a body tissue. The ultrasonic treatment device includes an ultrasonic transducer generating ultrasonic vibration and an ultrasonic probe configuring a treatment section. Ultrasonic vibration generated by the ultrasonic transducer is amplified and transmitted to the ultrasonic probe, where a clotting/incising treatment of a body tissue is performed utilizing the ultrasonic vibration.

As an ultrasonic transducer used in the ultrasonic treatment device, an apparatus having a bolt-tightening Langevin type transducer structure (for example, see U.S. Pat. No. 6,068,647 [Patent Document 1]) or an apparatus having a magnetostriction type transducer structure (for example, see U.S. Pat. No. 6,214,017 [Patent Document 2]) have been known. The bolt-tightening Langevin type transducer has such a structure that piezoelectric elements and electrodes are alternately stacked and the stacked body is arranged between a horn and a backing plate in a fastening manner. The magnetostriction type transducer has such a structure that magnetostriction material is wound by a coil.

Recently, as a candidate of material for artificial muscle, it is considered that silicon resin which is polymer material with electric field response called “dielectric elastomer” or electrostrictive polymer such as acrylic resin is used. For example, see Nikkei Science, February issue of 2004, pp. 56-65 (Non-Patent Document 1), Electronics Packaging Techniques 2002. 1 (Vol. 18 No. 1) pp. 32-38 (Non-Patent Document 2), and Mold Working: Vol. 16 No. 10 2004 pp. 631-637 (Non-Patent Document 3). Electrodes are formed on both face of the electrostrictive polymer in a thin film manner. When voltage is applied between both the electrodes, such an action that one face side is shrunk and the other face side is stretched occurs. In an actuator using such an electrostrictive polymer, the electrostrictive polymer shrinks and stretches to generate desired driving force according to cyclic application of voltage between the electrodes.

As a usage example of such an actuator utilizing an electrostrictive polymer, application to a prosthesis such as an artificial hand, an artificial leg, a haptic, a soft skin allowing sensing, or a pump for diagnosing blood or the like, or medical equipment has been researched (for example, see the abovementioned Non-Patent Document 3).

### BRIEF SUMMARY OF THE INVENTION

According to a first aspect of the present invention, an ultraeonic treatment device, comprising: at least a pair of electrodes disposed so as to be spaced from each other in an insulated state; an actuator main body formed from an electrostrictive polymer that is disposed between the respective electrodes, where the electrostrictive polymer is driven in a stretching and shrinking manner when voltage is applied between the respective electrodes; a power source means supplying voltage between the respective electrodes; and a treatment portion configured to treat a region to be treated by ultrasonic vibration thereof in linkage with driving of the electrostrictive polymer in the stretching and shrinking manner.

Preferably, the treatment portion includes a blade which is joined to the actuator and which generates ultrasonic vibration in linkage with driving of the actuator in the stretching and shrinking manner to conduct a treatment of the region to be treated, and the power source supplies AC voltage between the electrodes of the actuator.

Preferably, a positive electrode is disposed at one end portion of the actuator main body, and a negative electrode is disposed at the other end portion of the actuator main body.

Preferably, the actuator main body includes, therein, a plurality of positive internal electrodes and a plurality of negative internal electrodes embedded so as to extend approximately in parallel to a center axis direction of the actuator main body in a state the plurality of positive internal electrodes and the plurality of negative internal electrodes are provided approximately in parallel, one ends of the plurality of positive internal electrodes are connected to the positive electrode, one ends of the plurality of negative internal electrodes are connected to the negative electrode, and the plurality of positive internal electrodes and the plurality of negative internal electrodes are alternately provided at predetermined intervals in a paired manner.

Preferably, the actuator main body is formed from an electrostrictive polymer with a rectangular parallelepiped shape, a positive electrode is disposed on one side face of the rectangular parallelepiped shape of the actuator main body, and a negative electrode disposed on the other side face of the rectangular parallelepiped shape of the actuator main body.

Preferably, the actuator main body includes, therein, a plurality of positive internal electrodes and a plurality of negative internal electrodes embedded so as to extend approximately in parallel to a center axis direction of the actuator main body in a state the plurality of positive internal electrodes and the plurality of negative internal electrodes are provided approximately in parallel, one ends of the plurality of positive internal electrodes are connected to the positive electrode, one ends of the plurality of negative internal electrodes are connected to the negative electrode, and the plurality of positive internal electrodes and the plurality of negative internal electrodes are alternately provided at predetermined intervals in a paired manner.

Preferably, the actuator main body is formed of an electrostrictive polymer with a cylindrical shape, the pair of electrodes is disposed on an outer peripheral face and an inner peripheral face of the cylindrical shape of the actuator main body.

Preferably, the actuator main body includes a positive electrode disposed on an outer peripheral face of the cylindrical shape and a negative electrode disposed on an inner peripheral face of the cylindrical shape.

Preferably, the treatment portion includes a fixing shaft supporting the actuator, further comprising a joint structure including at least two sheets of film-like electrostrictive polymers, the electrostrictive polymers including positive electrodes formed on one faces thereof and negative electrodes formed on the other faces thereof, the joint structure being obtained by joining the two film-like electrostrictive polymers in a state where the negative electrodes of the two film-like electrostrictive polymers have been superimposed on each other, and the joint structure is wound on the fixing shaft spirally.

According to another aspect of the present invention, an ultrasonic treatment device comprising an insertion unit having a distal end portion and a proximal end portion and inserted into a channel of an endoscope, a treatment portion disposed at the distal end portion of the insertion unit and generating ultrasonic vibration to conduct a treatment of a region to be treated, and driving means for driving the treatment portion, the ultrasonic treatment device performing a treatment to the region to be treated by ultrasonic vibration of the treatment portion within an observation view field obtained by the endoscope, wherein the insertion unit includes a flexible sheath having a distal end portion and a proximal end portion, the distal end portion of the sheath is coupled with the treatment portion, an operation unit for operating the treatment portion is disposed at the proximal portion of the sheath, the driving means comprises an actuator provided with an actuator main body formed from an electrostrictive polymer and at least a pair of electrodes disposed so as to be spaced from each other in an insulated state from each other, where the electrostrictive polymer is driven in a stretching and shrinking manner when voltage is applied between the respective electrodes, and power source means supplying voltage between the respective electrodes of the actuator, and the treatment portion includes a blade which is joined to the actuator and which generates ultrasonic vibration in linkage with driving of the actuator in the stretching and shrinking manner to conduct a treatment of the site to be treated.

Preferably, the treatment portion includes a cylindrical blade having a distal end portion and a proximal end portion, the blade having a suction hole communicating with an inner cavity of the blade at the distal end portion, and the sheath includes a suction tube having a distal end portion and a proximal end portion and disposed in the sheath, the distal end portion of the tube communicating with an inner cavity of the blade, and the proximal end portion of the tube extending from the operation unit to the outside to be connected to a suction pump.

Preferably, the treatment portion comprises a cylindrical blade having a distal end portion and a proximal end portion, in which the driving means is housed, a distal end cover member having a distal end portion and a proximal end portion, the proximal end portion of the distal end cover member being coupled to the distal end portion of the sheath, a forceps piece rotatably coupled to the distal end cover member and supported to be openable and closable to the blade, a forceps piece driving unit driving the forceps piece in an opening and closing manner to the blade, an operation wire having a distal end portion and a proximal end portion, provided in the sheath so as to be movable in a center axial direction of the sheath, and operating the forceps piece driving unit, and a handle provided on the operation unit, driving the forceps piece driving unit via the operation wire, and driving the forceps piece in an opening and closing manner to the blade.

### BRIEF DESCRIPTION OF THE SEVERAL VIEWS OF THE DRAWING

FIG. 1 is a schematic configuration view of a main section showing a portion of an ultrasonic treatment device according to a first embodiment of the present invention in partially sectional manner;

FIG. 2 is a perspective view showing an actuator and a blade of the ultrasonic treatment device according to the first embodiment;

FIG. 3 is a vertical sectional view of the actuator and the blade shown in FIG. 2;

FIG. 4 is a schematic configuration view showing a relationship between the actuator and a power source of the ultrasonic treatment device according to the first embodiment;

FIG. 5A is a schematic configuration view of a main section shown for explaining a model of the actuator of the ultrasonic treatment device according to the first embodiment;

FIG. 5B is a schematic configuration view of a main section shown for explaining an operation principle of the model of the actuator of the ultrasonic treatment device according to the first embodiment;

FIG. 6 is a characteristic diagram showing a relationship between electric field E and strain of the model of the actuator shown in FIG. 5B;

FIG. 7 is a vertical sectional view showing a state where the actuator and a sheath of the ultrasonic treatment device according to the first embodiment have been covered with a resin tube;

FIG. 8 is a vertical sectional view showing a state where showing a state where the actuator of the ultrasonic treatment device according to the first embodiment have been covered with a resin tube;

FIG. 9 is a schematic configuration view showing a state where the ultrasonic treatment device according to the first embodiment has been inserted into a channel of an insertion unit of an endoscope;

FIG. 10 is a vertical sectional view showing a state where a blade and first and second cases, and sheathes of the ultrasonic treatment device according to the first embodiment have been inserted into the channel of the insertion unit of the endoscope in an enlarged manner;

FIG. 11 is a schematic configuration view of a main section showing a first modification example of the actuator of the ultrasonic treatment device according to the first embodiment;

FIG. 12 is an exploded perspective view showing a second modification example of the actuator of the ultrasonic treatment device according to the first embodiment;

FIG. 13 is a cross-sectional view showing an attaching state of the actuator of the second modification example;

FIG. 14 is a perspective view of a main section showing an attaching example of the actuator and a retaining member of the second modification example;

FIG. 15 is a schematic configuration view of a main section showing a third modification of the actuator of the ultrasonic treatment device according to the first embodiment;

FIG. 16 is a vertical sectional view of the actuator of the third modification example;

FIG. 17 is a perspective view showing a fourth modification example of the actuator of the ultrasonic treatment device according to the first embodiment;

FIG. 18 is a vertical sectional view of the actuator of the fourth modification example;

FIG. 19 is a schematic configuration view of a main section showing an ultrasonic suction apparatus according to a second embodiment of the present invention;

FIG. 20 is a vertical sectional view of an actuator of the ultrasonic suction apparatus according to the second embodiment;

FIG. 21 is a cross-sectional view of the actuator of the ultrasonic suction apparatus according to the second embodiment;

FIG. 22 is a vertical sectional view showing a state where outer peripheral faces of the actuator and a sheath of the ultrasonic suction apparatus according to the second embodiment have been covered with a resin tube for protection;

FIG. 23 is a vertical sectional view of a main section showing a modification example of the ultrasonic suction apparatus according to the second embodiment;

FIG. 24 is a schematic configuration view of a main section of an ultrasonic clotting/incising apparatus according to a third embodiment of the present invention;

FIG. 25 is a vertical sectional view showing a main section of the ultrasonic clotting/incising apparatus according to the third embodiment;

FIG. 26 is an exploded perspective view for explaining an arrangement relationship between a blade and an actuator of the ultrasonic clotting/incising apparatus according to the third embodiment;

FIG. 27 is a vertical sectional view of a main section showing a state where the actuator of the ultrasonic clotting/incising apparatus according to the third embodiment has been covered with a resin tube;

FIG. 28A is an explanatory diagram showing an initial position of the actuator of the ultrasonic clotting/incising apparatus according to the third embodiment; and

FIG. 28B is an explanatory diagram showing an operating state of the actuator of the ultrasonic clotting and incising apparatus according to the third embodiment.

### DETAILED DESCRIPTION OF THE INVENTION

Embodiments of the present invention will be explained in detail below with reference to the drawings.

### First Embodiment

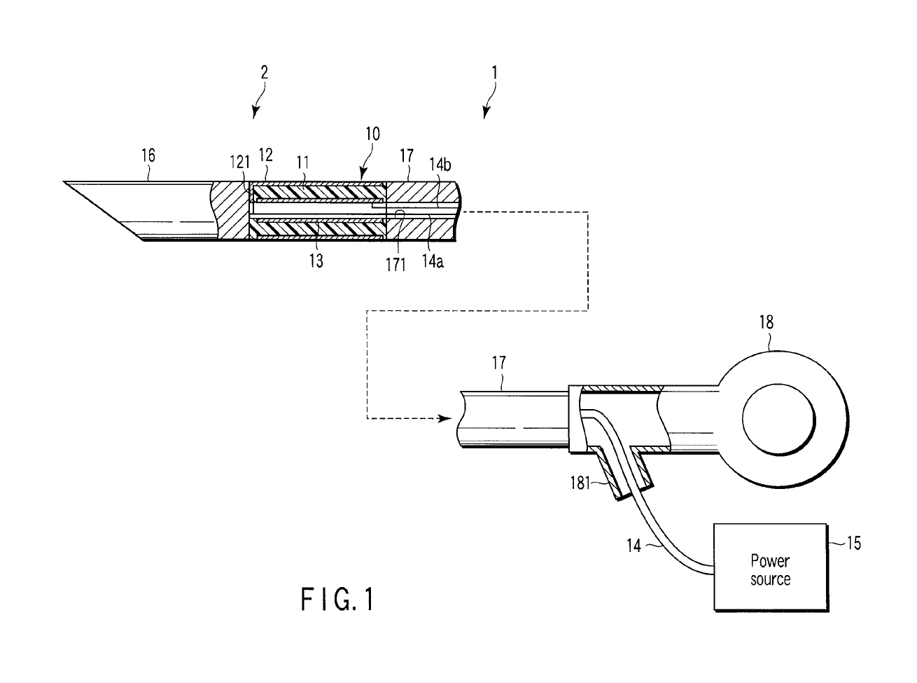

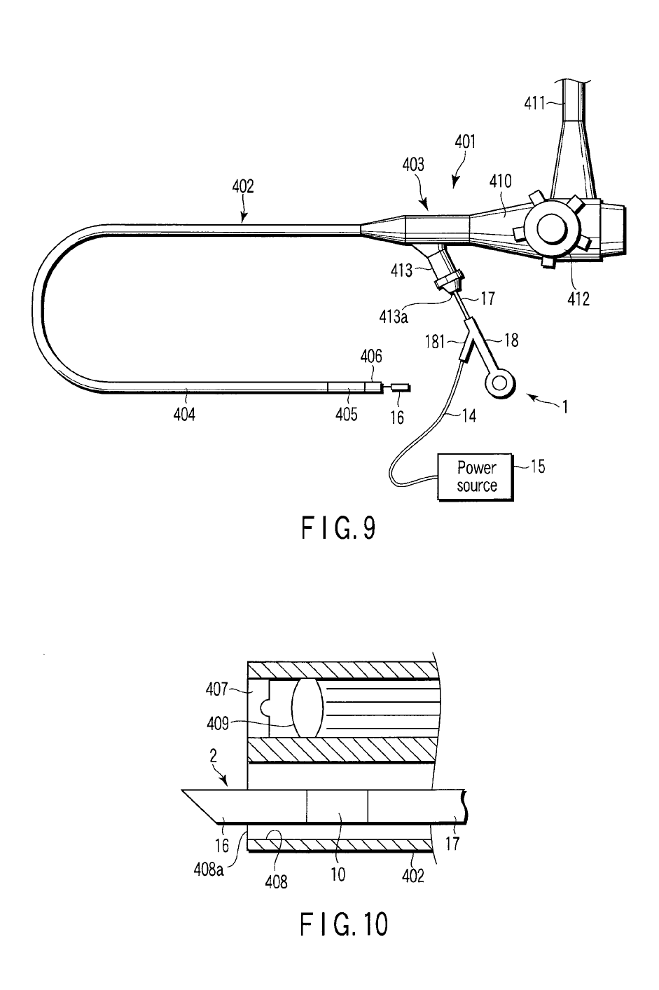

FIGS. 1 to 10 show a first embodiment of the present invention. FIG. 1 shows a schematic configuration of a main section of an ultrasonic treatment device 1 used together with an endoscope 401 (see FIG. 9).

FIG. 9 shows a schematic configuration of the endoscope 401. The endoscope 401 includes an elongated insertion unit 402 and an operation unit 403 connected to a proximal end portion of the insertion unit 402. The insertion unit 402 includes an elongated flexible pipe unit 404, a bending unit 405 whose proximal end portion is connected to a distal end portion of the flexible pipe unit 404, and a distal end hard portion 406 whose proximal end portion is coupled to a distal end portion of the bending unit 405. The bending unit 405 can be operated from its ordinary straight state extending straightly to a bent shape in a bending manner. As shown in FIG. 10, an illumination lens of an illumination optical system (not shown), an objective lens 407 of an observation optical system, a distal end opening portion 408a of a treatment device insertion channel 408, a gas/water feeding nozzle (not shown), and the like are disposed on a distal end face of the distal end hard portion 406.

An imaging device 409 such as a CCD, a connection circuit board for the imaging device 409, and the like are fixed at the distal end hard portion 406 behind the objective lens 407. Incidentally, a distal end portion of an image guide fiber (not shown) is fixed instead of the imaging device 409 and the endoscope 401 may be used for an electronic scope or a fiber scope may be used instead of the endoscope 401. Further, a distal end portion of the treatment device insertion channel 408, distal end portions of a gas feeding tube and a water feeding tube connected to the gas/water feeding nozzle and the like may be fixed at the distal end hard portion 406.

A handle portion 410 grasped by an operator is disposed on the operation unit 403. The handle portion 410 is connected with a proximal end portion of a universal cord 411. A connector portion connected to a light source apparatus (not shown) and a video processor are coupled to a distal end portion of the universal cord 411.

Further, a bending operation knob 412 operating the bending unit 405 in a bending manner, a suction button, a gas feeding/water feeding button, various switches for endoscope photographing, a treatment device insertion ferrule 413, and the like are provided on the operation unit 403. A treatment device insertion port 413a coupled to a proximal end portion of the treatment device insertion channel 408 disposed in the insertion unit 402 is provided in the treatment device insertion ferrule 413. After the ultrasonic treatment device 1 according to the embodiment which is a treatment device for an endoscope is inserted from the treatment device insertion port 413a of the endoscope 401 into the treatment device insertion channel 408 and it is operated up to the distal end hard portion 406 side in a pushing manner, it is protruded from the distal end opening portion 408a of the treatment device insertion channel 408 to the outside.

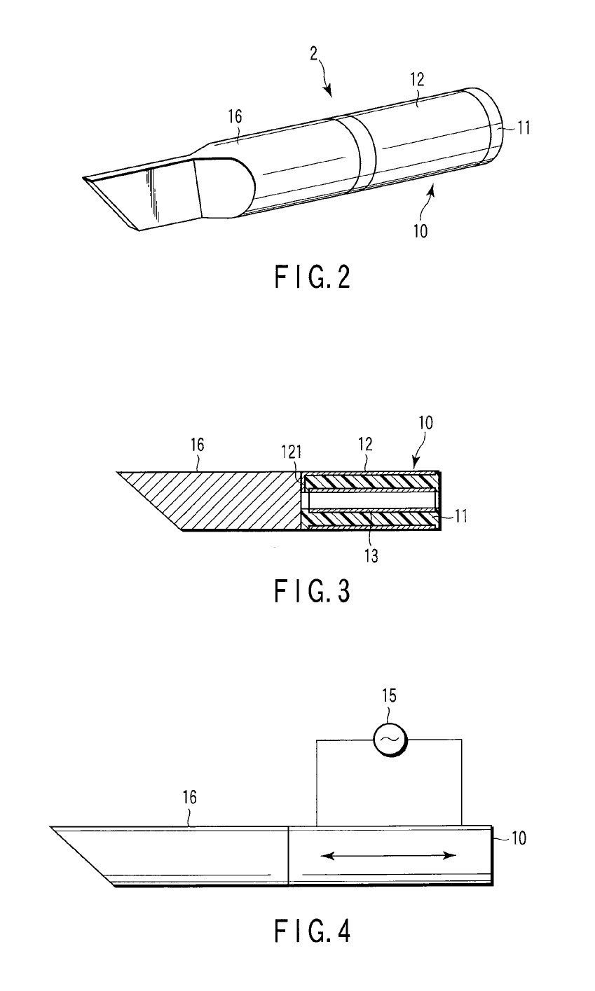

As shown in FIG. 1, the ultrasonic treatment device 1 includes an elongated flexible sheath 17 and an operating unit 18 coupled to the proximal end portion of the sheath 17. A distal end treatment portion 2 is coupled to a distal end portion of the sheath 17. As shown in FIG. 2, a blade 16 which is a treatment portion for performing a treatment such as incising/arrest of bleeding or the like of a body tissue and an actuator 10 configuring an ultrasonic transducer for conducting ultrasonic vibration of the blade 16 are provided at the distal end treatment portion 2.

A proximal end portion of the blade 16 is joined to a distal end portion of the actuator 10 utilizing a joining method such as, for example, adhesion, spot welding, or brazing.

As shown in FIG. 3, the actuator 10 includes a tube-like electrostrictive polymer 11, a stretchable and shrinkable positive electrode 12 disposed on an outer peripheral face of the electrostrictive polymer 11, and a stretchable and shrinkable negative electrode 13 disposed on an inner peripheral face of the electrostrictive polymer 11.

The electrostrictive polymer 11 is a capacitor having such elasticity as that of rubber, and it is called “dielectric elastomer” as shown in the abovementioned Non-Patent Document 2. The electrostrictive polymer 11 is formed in a tube-like shape made of such resin material as acrylic, silicon, or polyurethane. As the positive electrode 12 and the negative electrode 13, an electrode on which a film is formed, for example, using photolithography, a carbon electrode formed by mixing binder and carbon fine particles to spray the mixed material, or the like is used.

A positive electrode connecting terminal 121 bent toward an inner peripheral face side of the electrostrictive polymer 11 is formed at a distal end portion of the positive electrode 12 of the actuator 10. The positive electrode connecting terminal 121 extends up to an inner peripheral face of the electrostrictive polymer 11.

A wiring cable 14 is disposed in an inner hollow portion 171 of the sheath 17. A positive wire 14a and a negative wire 14b extend inside the wiring cable 14. A distal end portion of the positive wiring 14a is connected to the positive electrode connecting terminal 121, while a distal end portion of the negative wiring 14b is connected to the negative electrode 13.

A proximal end portion of the wiring cable 14 extends up to the operating unit 18 on a near side via the inner hollow portion 171 of the sheath 17. A cable pulling-out port 181 is provided on the operating unit 18 in a projecting manner. The wiring cable 14 is pulled out from the cable pulling-out port 181 to the outside. Further, the wiring cable 14 pulled out from the cable pulling-out port 181 is connected to a power source 15 configuring power source means. Voltage is applied from the power source 15 to the positive electrode 12 and the negative electrode 13 at a desired cycle.

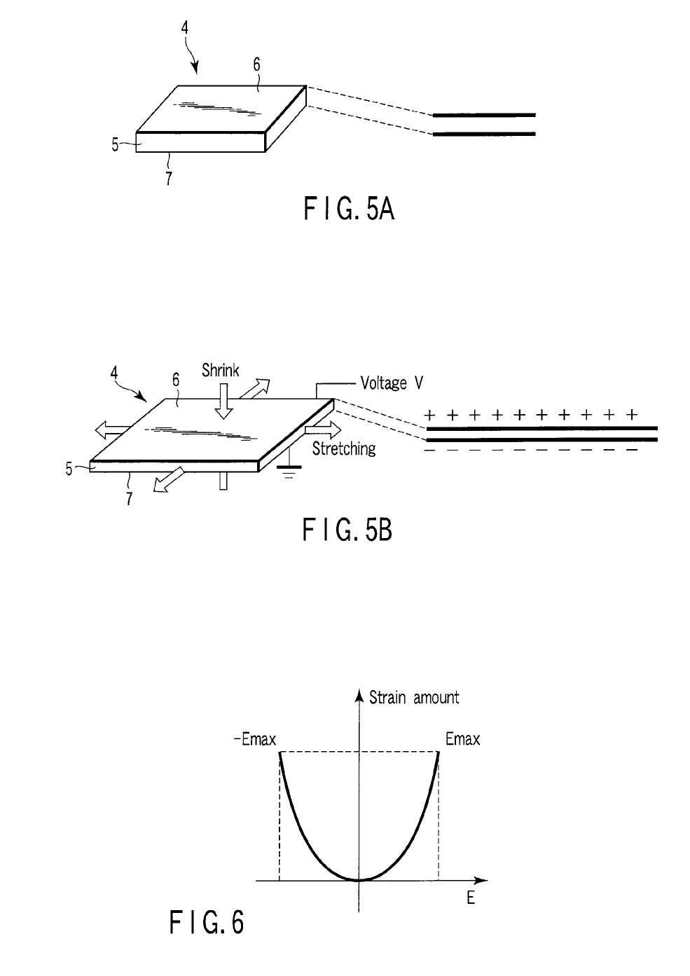

Next, a driving principle of the abovementioned actuator 10 will be explained with reference to FIGS. 5A, 5B, and 6. FIG. 5A shows a plate-like actuator model 4 utilizing an electrostrictive polymer, as shown in the previous mentioned Non-Patent Document 3. The actuator model 4 has a positive electrode 6 formed on one face of the plate-like electrostrictive polymer 5 and a negative electrode 7 formed on the other face thereof. Voltage is applied between the positive electrode 6 and the negative electrode 7 at a desired cycle. In this case, attracting force is generated between the positive electrode 6 and the negative electrode 7 of the electrostrictive polymer 5 in synchronism with the cycle of voltage. Therefore, as shown in FIG. 5B, the electrostrictive polymer 5 is shrunk in an inter-electrode direction and it is stretched in a direction orthogonal thereto. It has been confirmed that a strain coefficient is proportional to a square of electric field E applied as shown in FIG. 6 and it reaches several tens percentages to three hundreds percentages or more.

The abovementioned actuator 10 operates based upon the operation principle in the following manner. That is, voltage from the power source 15 is applied between the positive electrode 12 and the negative electrode 13 at a desired cycle at a time of actuation of the actuator 10. At this time, the electrostrictive polymer 11 generates attracting force between the positive electrode 12 and the negative electrode 13 in synchronism with the cycle of voltage as shown in the abovementioned Non-Patent Document 3. Thereby, the electrostrictive polymer 11 is shrunk in a direction orthogonal to a center line direction of the tube of the electrostrictive polymer 11 (a direction from an outer peripheral face toward an inner peripheral face of the electrostrictive polymer 11) and it is stretched in the center line direction of the tube. A shape change of the electrostrictive polymer 11 (shrinking and stretching behavior) is repeated in synchronism with the cycle of voltage so that ultrasonic vibration of the blade 16 is generated in linkage with the behavior.

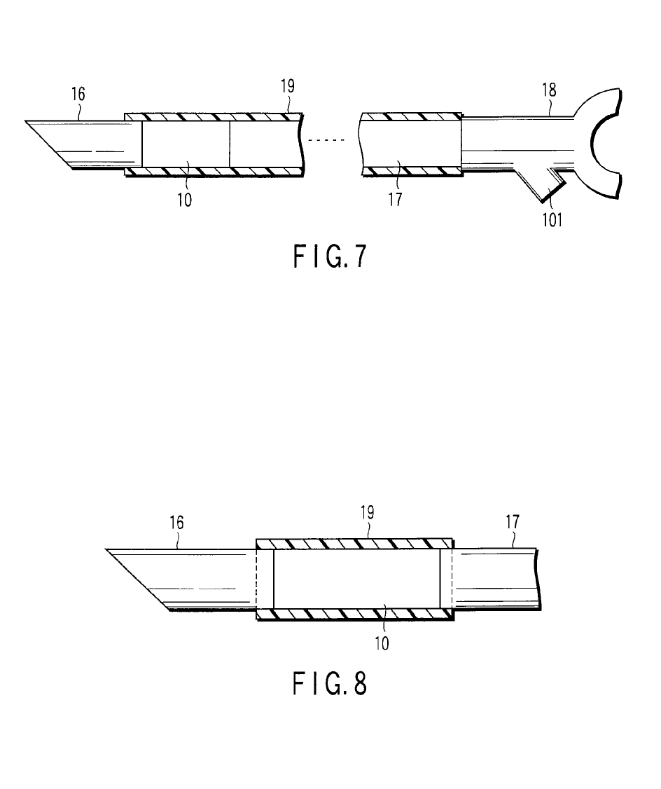

Outer peripheral faces of the abovementioned actuator 10 and sheath 17 are covered with a protection resin tube 19, for example, as shown in FIG. 7, and the tube 19 is fixed to the outer peripheral faces using adhesive. The protection resin tube 19 is made of resin material such as Teflon (registered trademark), polyethylene, acrylic, silicon, or polyurethane. As shown in FIG. 8, the resin tube 19 may be provided so as to cover only at least the actuator 10, as another way. Thereby, promotion of protection of the actuator 10 can be achieved.

As shown in FIG. 9, the abovementioned blade 16, actuator 10, and sheath 17 are inserted into the channel 408 of the insertion unit 402 via the ferrule 413 of the insertion unit 402 of the endoscope 401 disposed on the near side at a time of using the ultrasonic treatment device 1 according to the present embodiment. As shown in FIG. 10, the blade 16 of the distal end treatment portion 2 of the ultrasonic treatment device 1 is protruded from the distal end opening portion 408a of the treatment device insertion channel 408 to the outside. In this state, voltage is applied to the positive electrode 12 and the negative electrode 13 of the electrostrictive polymer 11 in the actuator 10 from the power source 15 at a desired cycle. Thereby, the electrostrictive polymer 11 is driven in the axial direction of the tube in a stretching and shrinking manner, thereby causing ultrasonic vibration of the blade 16. Therefore, it is made possible to conduct a treatment such as incising of a body tissue/arrest of bleeding by using the blade 16.

Next, an operation of the present embodiment with the abovementioned configuration will be explained. When a treatment such as incising of a body tissue/arrest of bleeding is conducted by using the ultrasonic treatment device with the abovementioned configuration, the insertion unit 402 of the endoscope 401 is first inserted into a body cavity. At this time, an observation image in the body cavity entered through the objective lens 407 of the observation optical system of the endoscope 401 is picked up by the imaging device 409, the image is observed through a screen displayed on a monitor (not shown), and an affected area is confirmed. In a state that the affected area has been confirmed by the endoscope 401, the ultrasonic treatment device 1 is inserted into the treatment device insertion channel 408 through the treatment device insertion ferrule 413 of the operation unit 403 of the endoscope 401. As shown in FIG. 10, the blade 16 of the distal end treatment portion 2 of the ultrasonic treatment device 1 is protruded from the distal end opening portion 408a of the treatment device insertion channel 408 to the outside.

Next, the operating unit 18 is operated while the affected area is observed by the endoscope 401, so that the blade 16 is adjusted to a treatment site while it is moved back and forth. Thereafter, ultrasonic generation operation means (not shown) of the ultrasonic treatment device 1, for example, a foot switch, a hand switch, or the like is operated. Here, voltage is applied to the positive electrode 12 and the negative electrode 13 of the actuator 10 at a desired cycle. Thereby, in the actuator 10, the electrostrictive polymer 11 is stretched and shrunk in the axial direction of the tube in synchronism with a supplying cycle of voltage from the power source 15. Therefore, the blade 16 is subject to ultrasonic oscillation so that incising of a body tissue/arrest of bleeding treatment is performed.

With the abovementioned configuration, the following effects can be obtained. That is, the ultrasonic treatment device 1 is configured such that the positive electrode 12 and the negative electrode 13 are disposed on the electrostrictive polymer 11, the actuator 10 where the electrostrictive polymer 11 is driven in the stretching and shrinking manner when voltage is applied between the electrodes 12 and 13 is provided, and the blade 16 is ultrasonically vibrated by supplying voltage to the positive electrode 12 and the negative electrode 13 of the actuator 10 at a predetermined cycle to drive the electrostrictive polymer 11 in a stretching and shrinking manner. Thereby, the blade 16 can be ultrasonically vibrated with large amplitude, so that the blade 16 having high treatment ability can be realized using a small-sized actuator 10 and size-reduction of an apparatus can be achieved.

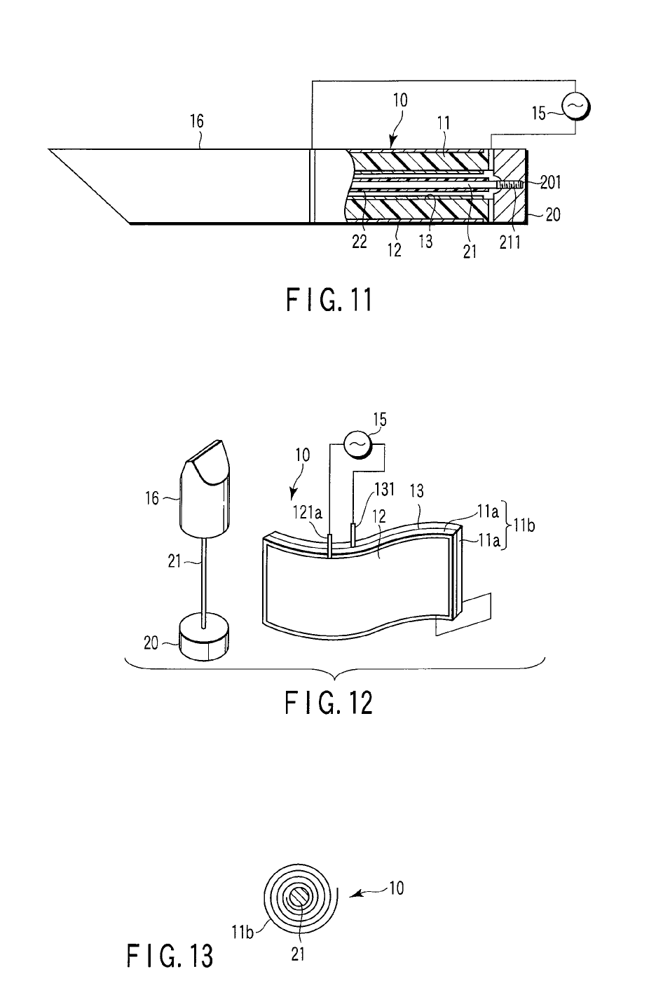

FIG. 11 shows a first modification example of the actuator 10 of the ultrasonic treatment device 1 according to the first embodiment (see FIG. 1 to FIG. 10). In the abovementioned embodiment, the configuration where the electrostrictive polymer 11 of the actuator 10 is joined to the blade 16 by a method such as adhesion, spot welding, or brazing has been shown, but the present invention is not limited to this configuration. In the present modification example, attaching means for attaching the electrostrictive polymer 11 of the actuator 10 to the blade 16 is modified in the following manner.

In the present modification, a fixing shaft 21 inserted into the electrostrictive polymer 11 is provided on the blade 16 in a projecting manner. A distal end portion of the fixing shaft 21 is formed with a male screw unit 211. A retaining member 20 is disposed on a rear end face of the electrostrictive polymer 11 of the actuator 10. The retaining member 20 is provided with a screw hole 201.

Such a configuration is adopted that the electrostrictive polymer 11 of the actuator 10 is fixed to the blade 16 by screwing the screw unit 211 of the fixing shaft 21 of the blade 16 into the screw hole 201 of the retaining member 20. In this case, an effect similar to that obtained in the first embodiment can be obtained.

Incidentally, when the fixing shaft 21 is made of, for example, metal with electrical conductivity or the like, such a configuration can be adopted that a periphery of the fixing shaft 21 is covered with an insulating tube 22.

FIGS. 12 to 14 show a second modification example of the actuator 10 of the ultrasonic treatment device 1 of the first embodiment (see FIG. 1 to FIG. 10). In the present modification example, the configuration where the electrostrictive polymer 11 of the actuator 10 is fixed to the blade 16 using the fixing shaft 21 and the retaining member 20 according to the first modification example (see FIG. 11) is modified in the following manner.

That is, in the present modification example, at least two sheets of film-like electrostrictive polymers 11a are used, as shown in FIG. 12. One face of the film-like electrostrictive polymer 11a is formed with a positive electrode 12 and the other face thereof is formed with a negative electrode 13, respectively. Two sheets of film-like electrostrictive polymers 11a are joined to each other in a state that their negative electrodes 13 have been stacked, for example. Thereby, a joint structure 11b obtained by joining two sheets of film-like electrostrictive polymers 11a is formed.

As shown in FIG. 13, the joint structure 11b is wound on the fixing shaft 21 in a spiral manner. Thereby, the actuator 10 is formed between the blade 16 and the retaining member 20.

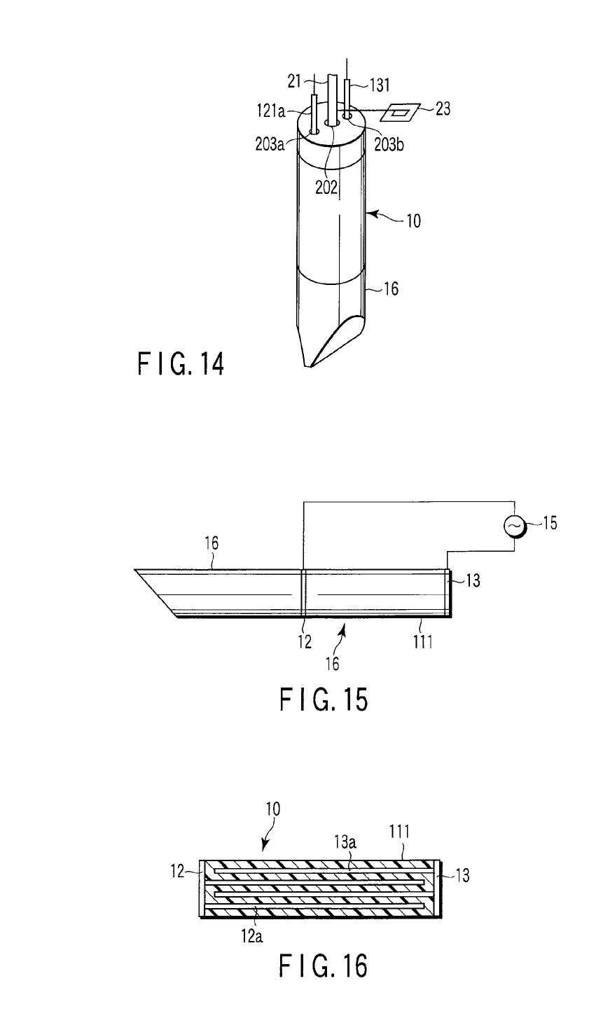

In the actuator 10 according to the present modification, as shown in FIG. 14, a fixing shaft insertion hole 202 and a pair of electrode insertion holes 203a, 203b are provided in a rear end portion of the retaining member 20. The fixing shaft insertion hole 202 is disposed at an axial center portion of the rear end portion of the retaining member 20. The fixing shaft 21 fixed to the blade 16 is inserted into the fixing shaft insertion hole 202. A fixing member 23 is attached to an end portion of the fixing shaft 21 to be fixed to the retaining member 20.

On the other hand, a positive electrode connection terminal 121a connected to the positive electrode 12 is inserted into one electrode insertion holes 203a. A negative electrode connecting terminal 131 (see FIG. 12) connected to the negative electrode 13 is inserted into the other electrode insertion hole 203b. Further, the positive electrode connecting terminal 121a is connected to the positive wire 14a of the wiring cable 14, while the negative electrode connecting terminal 131 is connected to the negative wire 14b of the wiring cable 14. The positive electrode connecting terminal 121a and the negative electrode connection terminal 131 are connected to the power source 15 via the wiring cable 14.

In the present modification example, it is possible to form the electrostrictive polymer 11a in a state that it has been stretched in advance so that it is made possible to set a strain obtained when voltage is applied to the positive electrode 12 and the negative electrode 13 to be large. Therefore, it is possible to further improve the strain coefficient.

Incidentally, as shown in the abovementioned Non-Patent Document 1, a spiral spring may be used instead of the fixing shaft 21, but rigidity can be increased by using the fixing shaft 21, so that the frequency characteristic can be further improved.

FIGS. 15 and 16 show a third modification example of the actuator 10 of the ultrasonic treatment device 1 according to the first embodiment (see FIGS. 1 to 10). In the abovementioned first embodiment, the configuration where the tube-like member is used as the electrostrictive polymer 11 of the actuator 10 has been shown, but the present invention is not limited to this configuration. In the present modification example, the electrostrictive polymer 11 has been modified in the following manner. Incidentally, in FIGS. 15 and 16, same portions as those in FIGS. 1 to 10 are attached with same reference numbers and detailed explanation thereof is omitted.

That is, in the present modification example, an electrostrictive polymer 111 formed in a rectangular shape is provided, as shown in FIG. 15. One end face of the electrostrictive polymer 111 is provided with a positive electrode 12 and the other face thereof is provided with a negative electrode 13, respectively. The positive electrode 12 and the negative electrode 13 are connected to the abovementioned power source 15.

FIG. 16 shows an internal configuration of the electrostrictive polymer 111. A plurality of positive internal electrodes 12a and a plurality of negative internal electrodes 13a are embedded in the electrostrictive polymer 111. The plurality of positive internal electrodes 12a and the plurality of negative internal electrodes 13a are provided approximately in parallel to one another in state that they extend approximately in parallel with a center line of the electrostrictive polymer 111. One end portions of the plurality of positive internal electrodes 12a are connected to the positive electrode 12. Similarly, one end portions of the plurality of negative internal electrodes 13a are connected to the negative electrode 13. Further, the plurality of positive internal electrodes 12a and the plurality of negative internal electrodes 13a are alternately provided at predetermined intervals in a paired manner.

In the present modification example, an effect similar to that of the first embodiment is obtained.

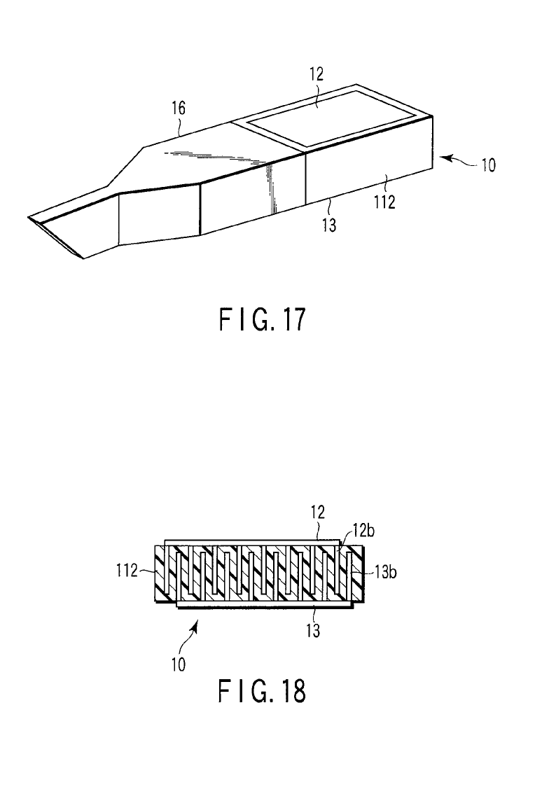

FIGS. 17 and 18 show a fourth modification example of the actuator 10 of the ultrasonic treatment device 1 according to the first embodiment. In the present modification example, a rectangular electrostrictive polymer 112 is formed, as shown in FIG. 17. A positive electrode 12 is disposed on an upper face of the rectangular electrostrictive polymer 112, while a negative electrode 13 is disposed on a lower face of the rectangular electrostrictive polymer 112. The positive electrode 12 and the negative electrode 13 are connected to the abovementioned power source 15.

FIG. 18 shows an internal configuration of the electrostrictive polymer 112. A plurality of positive internal electrodes 12b and a plurality of negative internal electrodes 13b are embedded in the electrostrictive polymer 112. The plurality of positive internal electrodes 12b and the plurality of negative internal electrodes 13b are provided approximately in parallel to one another in state that they extend in a direction approximately perpendicular to a center line of the electrostrictive polymer 112. One end portions of the plurality of positive internal electrodes 12b are connected to the positive electrode 12. Similarly, one end portions of the plurality of negative internal electrodes 13b are connected to the negative electrode 13. Further, the plurality of positive internal electrodes 12b and the plurality of negative internal electrodes 13b are alternately provided at predetermined intervals in a paired manner.

In the present modification example, an effect similar to that of the first embodiment is obtained.

### Second Embodiment

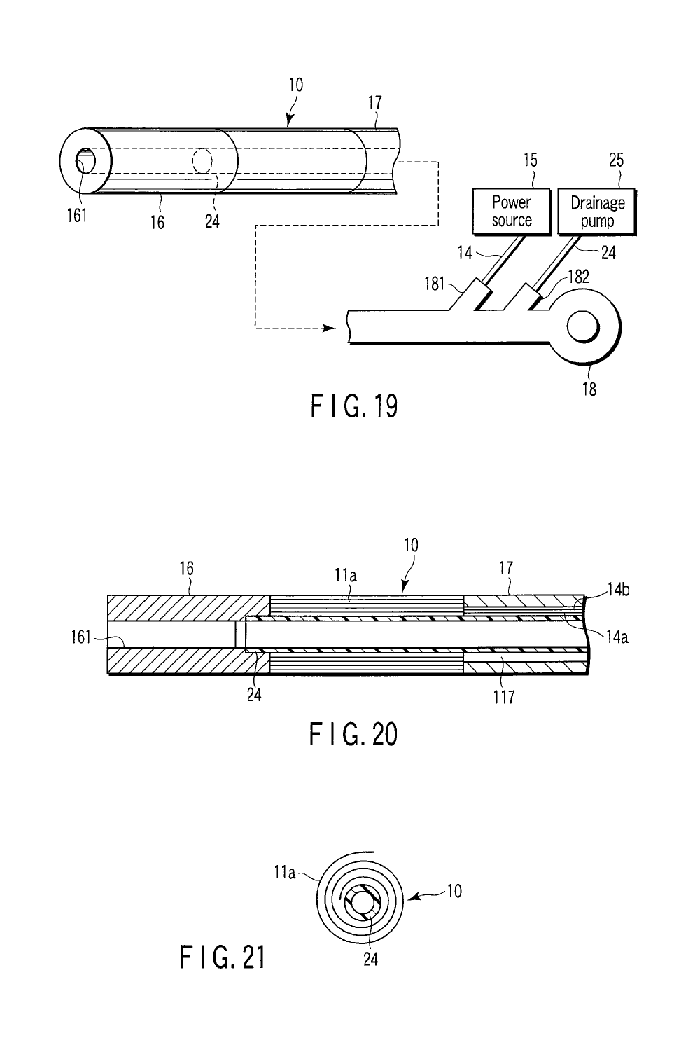

FIGS. 19 to 23 show a second embodiment of the present invention. The present embodiment shows an example where the present invention has been applied to an ultrasonic suction apparatus 1A performing crushing and emulsifying treatment of a body issue and serving as the ultrasonic treatment device 1. In FIGS. 19 to 23, same portions as those in the first embodiment (see FIGS. 1 to 10) are attached with same reference numbers and detailed explanation thereof is omitted.

That is, in the present embodiment, as shown in FIGS. 19 and 20, a through-hole 161 is provided at an axial center portion of a blade 16 of a distal end treatment portion 2. In an actuator 10 according to the present embodiment, an electrostrictive polymer 11a having the same configuration as that of the second modification example (see FIGS. 12 to 14) of the first embodiment is used.

As shown in FIG. 20, a suction tube 24 is inserted into the sheath 17. After a distal end portion of the tube 24 is inserted into an inner hollow portion of the electrostrictive polymer 11a of the actuator 10, it is fixed in a state that it has been inserted in a through-hole 161 of the blade 16.

As shown in FIG. 19, a tube discharging port 182 is provided in an operating unit 18 adjacent to a cable pulling-out port 181. A proximal end portion of the tube 24 is pulled cut from the tube discharging port 182 of the operating unit 18 to be coupled to a drainage pump 25.

A wiring cable 14 connected to a positive electrode connection terminal 121a connected to a positive electrode 12 of the actuator 10 and a negative electrode connection terminal 131 connected to a negative electrode 13 thereof, respectively is provided so as extend up to the operating unit 18 a gap through between an inner hollow portion 171 of the sheath 17 and the tube 24. The wiring cable 14 is pulled out from the cable pulling-out port 181 to be connected to the power source 15.

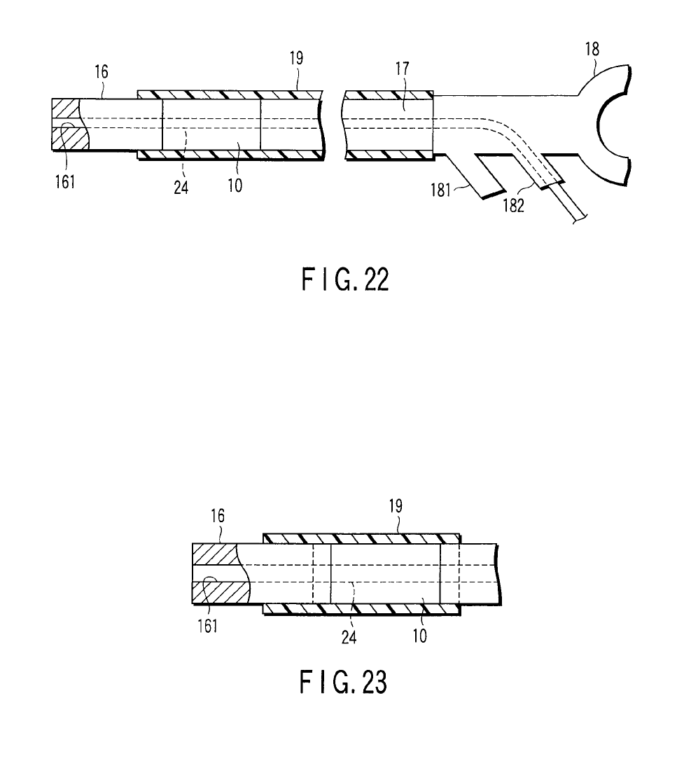

Outer peripheral faces of the actuator 10 and the sheath 17 are covered with a protection resin tube 19, for example, as shown in FIG. 22, and the protection resin tube 19 is fixed to the outer peripheral faces, for example, by using adhesive. The protection resin tube 19 is made of resin material such as Teflon (registered trademark), polyethylene, acrylic, silicon, or polyurethane like the abovementioned first embodiment. Like a modification example shown in FIG. 23, the resin tube 19 may be provided so as to cover only at least the actuator 10, as another way. Thereby, promotion of protection of the actuator 10 can be achieved.

Next, an operation of the ultrasonic suction apparatus 1A of the present embodiment with the abovementioned configuration will be explained. When a crushing/emulsifying treatment of a body tissue is performed by using the ultrasonic suction apparatus 1A with the abovementioned configuration, the insertion unit 402 of the endoscope 401 is first inserted into a body cavity. At this time, an observation image in the body cavity entered through the objective lens 407 of the observation optical system of the endoscope 401 is picked up by the imaging device 409, the image is observed through a screen displayed on a monitor (not shown), and an affected area is confirmed. In a state that the affected area has been confirmed by the endoscope 401, the ultrasonic suction apparatus 1A is inserted into the treatment device insertion channel 408 through the treatment device insertion ferrule 413 of the operation unit 403 of the endoscope 401. The blade 16 of the distal end treatment portion 2 of the ultrasonic suction apparatus 1A with the abovementioned configuration is protruded from the distal end opening portion 408a of the treatment device insertion channel 408 to the outside (see FIG. 10).

Next, the operating unit 18 is operated while the affected area is being observed, so that the blade 16 is moved back and forth. Thereby, the distal end portion of the blade 16 is caused to approach in accordance with a treatment side. Thereafter, ultrasonic generation operating means (not shown) is operated. Here, voltage from the power source 15 is applied to the positive electrode 12 and the negative electrode 13 of the actuator 10 at a desired cycle. Thereby, in the actuator 10, the electrostrictive polymer 11a is stretched and shrunk in an axial direction of the tube in synchronism with a supplying cycle of voltage from the power source 15. Thereby, the blade 16 is ultrasonically vibrated so that a crushing/emulsifying treatment of a body tissue is performed.

At this time, the drainage pump 25 is driven. Therefore, the body tissue which has been subjected to the crushing/emulsifying treatment is sucked into the through-hole 161 of the blade 16 via the tube 24 to be drained from the drainage pump 25 to a discharging bottle (not shown).

Therefore, the following effect can be obtained in the apparatus with the abovementioned configuration. That is, in the ultrasonic suction apparatus 1A according to the embodiment, such a configuration is adopted that the positive electrode 12 and the negative electrode 13 are disposed in the electrostrictive polymer 11a and the actuator 10 where the electrostrictive polymer 11a is driven in a stretching and shrinking manner when voltage is applied between the electrodes 12 and 13 is provided so that the blade 16 is ultrasonically vibrated by supplying voltage to the positive electrode 12 and the negative electrode 13 of the actuator 10 at a predetermined cycle to drive the electrostrictive polymer 11a in a stretching and shrinking manner. Therefore, the ultrasonic suction apparatus 1A which has a simple configuration like the first embodiment and can achieve promotion of size reduction can be provided.

### Third Embodiment

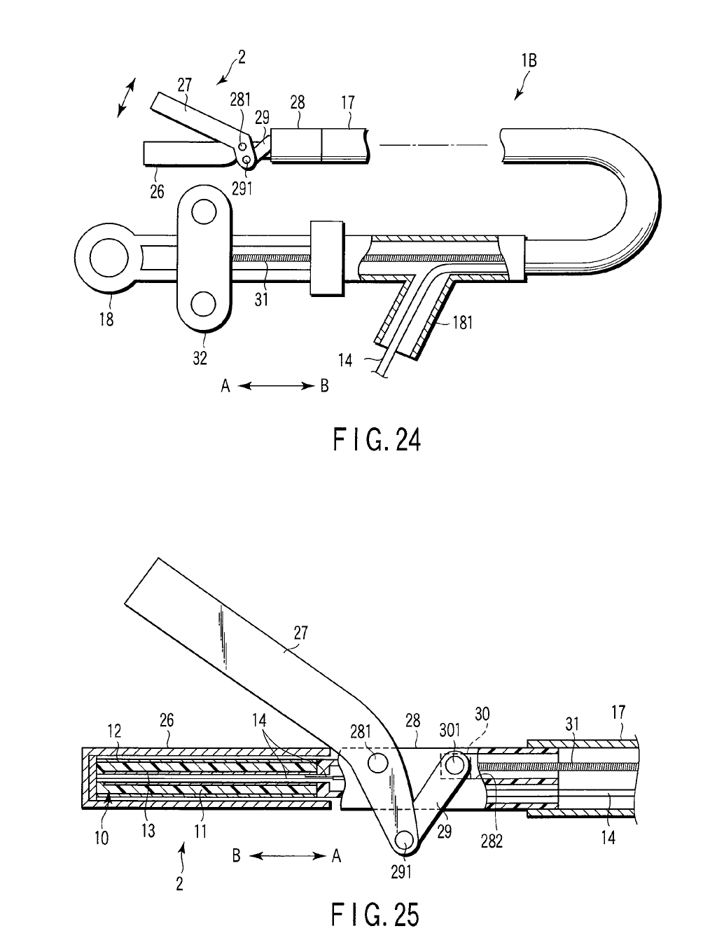

FIGS. 24 to 28B show a third embodiment of the present invention. The present embodiment shows an example that the present invention has been applied to an ultrasonic clotting/incising apparatus 1B which clips a body tissue to perform a clotting/incising treatment and serves as the ultrasonic treatment device. In FIGS. 24 to 28B, same portions as those in the first embodiment (see FIGS. 1 to 10) are attached with same reference numbers and detailed explanation thereof is omitted.

That is, in the present embodiment, a forceps piece 27 is disposed at a proximal end portion of a blade 26 disposed at a distal end treatment portion 2 in an openable and closable manner.

A proximal end portion of a cylindrical distal end cover 28 is fixed at a distal end portion of such a sheath 17 as a tightly wound coil. As shown in FIG. 25, an intermediate portion of the forceps piece 27 is rotatably pivoted to the distal end cover 28 via a main shaft pin 281.

A distal end portion of a link member 29 is rotatably attached to a proximal end portion of the forceps piece 27 via a supporting pin 291. A coupling member 30 is rotatably attached to a proximal end portion of the link member 29 via an operation pin 301.

The coupling member 30 is disposed so as to be movable in an axial direction along a guide groove 282 provided at the distal end cover 28. A distal end portion of an operation wire 31 is attached to the coupling member 30. The operation wire 31 is inserted into the abovementioned sheath 17.

A handle 32 is attached to the operating unit 18 so as to be operable in an axial direction of the operation wire 31 in a sliding manner. A proximal end portion of the operation wire 31 is fixed to the handle 32. In FIG. 24, the forceps piece 27 is operated so as to be moved via the operation wire 31 according to a sliding operation of the handle 32 in directions of arrows A and B.

When the handle 32 is operated toward the near side in a pulling manner so that the operation wire 31 is pulled in a direction of arrow A in FIG. 15, the link member 29 is operated toward the near side via the coupling member 30 and the operation pin 301 in a pulling manner to be rotated in a clockwise direction. Thereby, the forceps piece 27 is rotated about the main shaft pin 281 in a counterclockwise direction. As a result, the forceps piece 27 is moved in a direction of approaching the blade 26 (in a closing direction).

When the handle 32 is operated in a pushing-out direction, the operation wire 31 is moved in a direction of arrow B in FIG. 24. At this time, the link member 29 is operated via the coupling member 30 and the operation pin 301 in a pushing-out manner to be rotated in a counterclockwise direction. Thereby, the forceps piece 27 is rotated about the main shaft pin 281 in a clockwise direction. As a result, the forceps piece 27 is moved in a direction in which it is separated from the blade 26 (in an opening direction).

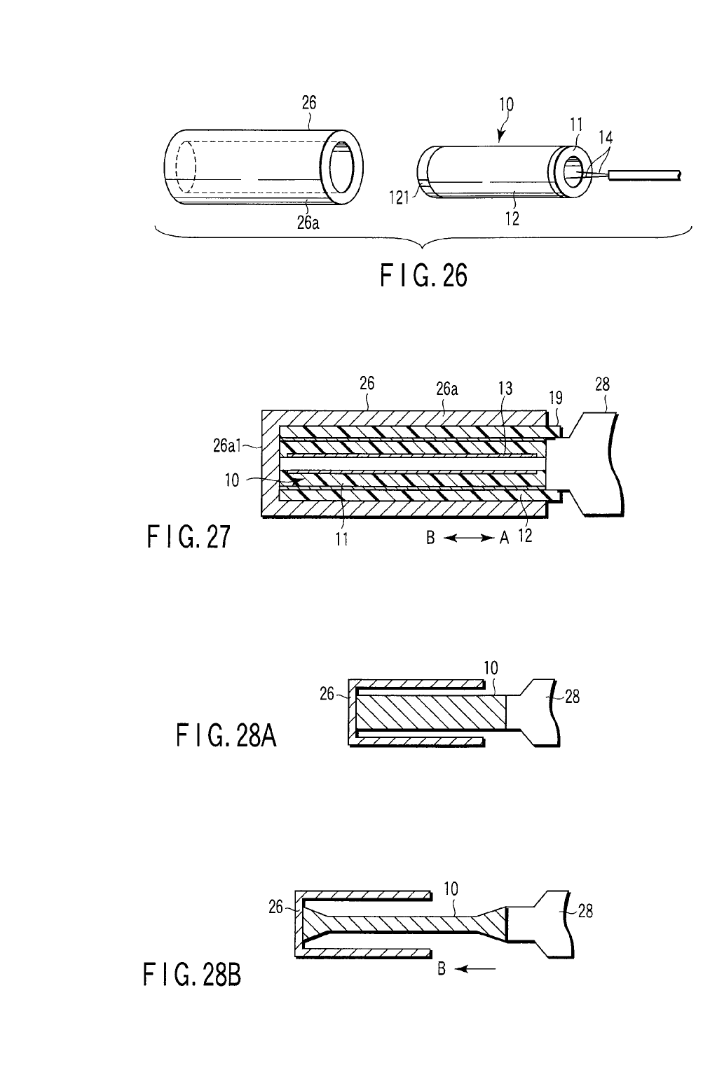

As shown in FIGS. 26 and 27, the blade 26 has a blade main body 26a formed in a cylindrical shape with a bottom. A cylindrical distal end of the blade main body 26a is closed by an end plate 26a1. A cylindrical actuator 10 is accommodated and disposed in a cylindrical inner hollow portion of the blade main body 26a.

The actuator 10 includes a cylindrical electrostrictive polymer 11, a positive electrode 12 attached on an outer peripheral face of the electrostrictive polymer 11, and a negative electrode 13 attached on an inner peripheral face of the electrostrictive polymer 11. A distal end portion of the electrostrictive polymer 11 of the actuator 10 is attached to the end plate 26a1 of the blade main body 26a in a state that the former has been caused to abut on the latter. A distal end portion of the distal end cover 28 is attached to a proximal end portion of the actuator 10 inserted into the blade 26. Thereby, the actuator 10 accommodated and disposed in the blade 26 is disposed between the end plate 26a1 of the blade 26 and the distal end portion of the distal end cover 28.

The actuator 10 can take any configuration explained regarding the ultrasonic treatment device 1 according to the first embodiment, where a similar effect can be obtained.

An outer peripheral face of the actuator 10 inserted into the blade 26 is covered with a protection resin tube 19, for example, as shown in FIG. 27, where the former is fixed to the latter, for example, by using adhesive. The protection resin tube 19 is made from resin material such as Teflon (registered trademark), polyethylene, acrylic, silicon, or polyurethane similar to the first and the second embodiments. Thereby, promotion of protection of the actuator 10 can be achieved.

The wiring cable 14 connected to the positive electrode 12 and the negative electrode 13 of the actuator 10 is inserted into the distal end cover 28 and the wiring cable 14 is connected to the abovementioned power source 15. Thereby, voltage from the power source 15 is applied between the positive electrode 12 and the negative electrode 13 via the wiring cable 14, and the actuator 10 is deformed between an initial position shown in FIG. 28A and an stretched position where the actuator 10 is stretched in a direction of arrow B (in an axial direction) as shown in FIG. 28B according to a cycle of the application.

Next, an operation of the present embodiment with the abovementioned configuration will be explained. In the ultrasonic clotting/incising apparatus 1B with the abovementioned configuration, when a clotting and incising treatment of a body tissue is performed, the insertion unit 402 of the endoscope 401 is first inserted into a body cavity. At this time, an observation image in the body cavity entered through the objective lens 407 of the observation optical system of the endoscope 401 is picked up by the imaging device 409, the image is observed through a screen displayed on a monitor (not shown), and an affected area is confirmed. In a state that the affected area has been confirmed by the endoscope 401, the ultrasonic clotting and incising apparatus 1B is inserted into the treatment device insertion channel 408 through the treatment device insertion ferrule 413 of the operation unit 403 of the endoscope 401. The blade 26 of the distal end treatment portion 2 of the ultrasonic clotting and incising apparatus 1B with the abovementioned configuration is protruded from the distal end opening portion 408a of the treatment device insertion channel 408 to the outside (see FIG. 6).

Next, the operating unit 18 is operated while the affected area is being observed, so that the blade 26 is moved back and forth. Thereby, the distal end portion of the blade 26 is caused to approach in accordance with a treatment side. Thereafter, the handle 32 of the operating unit 18 is operated in a pushing-out manner so that the forceps piece 27 is opened. In this state, movement and adjustment are performed such that an affected area is positioned between the blade 26 and the forceps piece 27. Subsequently, after such a state that the affected area has been positioned between the blade 26 and the forceps piece 27 is confirmed, the handle 32 is operated in a pulling manner. Thereby, the forceps piece 27 is rotated in a closing direction so that a body tissue is held between the forceps piece 27 and the blade 26.

Next, while the holding state is being confirmed, the operating unit 18 is operated and voltage of the power source 15 is applied to the positive electrode 12 and the negative electrode 13 of the actuator 10 at a desired cycle. Thereby, the electrostrictive polymer 11 of the actuator 10 is deformed in a stretching and shrinking manner in the axial direction between the initial position shown in FIG. 28A and the stretched position where it is stretched in the direction of arrow B (in the axial direction) as shown in FIG. 28B in synchronism with a supplying cycle of voltage from the power source 15. Thereby, the blade 26 is ultrasonically vibrated so that a clotting and incising treatment of the body tissue held between the blade 26 and the forceps piece 27 is performed.

As a result, the following effect can be achieved in the apparatus with the abovementioned configuration. That is, in the ultrasonic clopping and incising apparatus 1B according to the present embodiment, a blade 26 with a high treatment ability can be realized by using the actuator 10 using the electrostrictive polymer 11 so that an ultrasonic clopping and incising apparatus 1B reduced in size and having high treatment ability can be provided.

Incidentally, the present invention is not limited to the abovementioned embodiments and it can be modified and implemented variously without departing from the gist of the present invention. Further, inventions at various stages are included in the abovementioned embodiments, where various inventions can be extracted according to proper combinations in a plurality of constituent elements disclosed.

What is claimed is:

1. An ultraeonic treatment device, comprising:
at least a pair of electrodes disposed so as to be spaced from each other in an insulated state;
an actuator main body formed from an electrostrictive polymer that is disposed between the respective electrodes, where the electrostrictive polymer is driven in a stretching and shrinking manner when voltage is applied between the respective electrodes;
a power source means supplying voltage between the respective electrodes; and
a treatment portion configured to treat a region to be treated by ultrasonic vibration thereof in linkage with driving of the electrostrictive polymer in the stretching and shrinking manner.

2. The ultrasonic treatment device according to claim 1, wherein
the treatment portion includes a blade which is joined to the actuator and which generates ultrasonic vibration in linkage with driving of the actuator in the stretching and shrinking manner to conduct a treatment of the region to be treated, and
the power source supplies AC voltage between the electrodes of the actuator.

3. The ultrasonic treatment device according to claim 1, wherein
a positive electrode is disposed at one end portion of the actuator main body, and
a negative electrode is disposed at the other end portion of the actuator main body.

4. The ultrasonic treatment device according to claim 3, wherein
the actuator main body includes, therein, a plurality of positive internal electrodes and a plurality of negative internal electrodes embedded so as to extend approximately in parallel to a center axis direction of the actuator main body in a state the plurality of positive internal electrodes and the plurality of negative internal electrodes are provided approximately in parallel,
one ends of the plurality of positive internal electrodes are connected to the positive electrode,
one ends of the plurality of negative internal electrodes are connected to the negative electrode, and
the plurality of positive internal electrodes and the plurality of negative internal electrodes are alternately provided at predetermined intervals in a paired manner.

5. The ultrasonic treatment device according to claim 1, wherein
the actuator main body is formed from an electrostrictive polymer with a rectangular parallelepiped shape,
a positive electrode is disposed on one side face of the rectangular parallelepiped shape of the actuator main body, and
a negative electrode is disposed on the other side face of the rectangular parallelepiped shape of the actuator main body.

6. The ultrasonic treatment device according to claim 5, wherein
the actuator main body includes, therein, a plurality of positive internal electrodes and a plurality of negative internal electrodes embedded so as to extend approximately in parallel to a center axis direction of the actuator main body in a state the plurality of positive internal electrodes and the plurality of negative internal electrodes are provided approximately in parallel,
one ends of the plurality of positive internal electrodes are connected to the positive electrode,
one ends of the plurality of negative internal electrodes are connected to the negative electrode, and
the plurality of positive internal electrodes and the plurality of negative internal electrodes are alternately provided at predetermined intervals in a paired manner.

7. The ultrasonic treatment device according to claim 1, wherein
the actuator main body is formed of an electrostrictive polymer with a cylindrical shape,
the pair of electrodes is disposed on an outer peripheral face and an inner peripheral face of the cylindrical shape of the actuator main body.

8. The ultrasonic treatment device according to claim 7, wherein
the actuator main body includes a positive electrode disposed on an outer peripheral face of the cylindrical shape and a negative electrode disposed on an inner peripheral face of the cylindrical shape.

9. The ultrasonic treatment device according to claim 1, wherein
the treatment portion includes a fixing shaft supporting the actuator,
further comprising a joint structure including at least two sheets of film-like electrostrictive polymers, the electrostrictive polymers including positive electrodes formed on one faces thereof and negative electrodes formed on the other faces thereof, the joint structure being obtained by joining the two film-like electrostrictive polymers in a state where the negative electrodes of the two film-like electrostrictive polymers have been superimposed on each other, and
the joint structure is wound on the fixing shaft spirally.

10. An ultrasonic treatment device comprising
an insertion unit having a distal end portion and a proximal end portion and inserted into a channel of an endoscope,
a treatment portion disposed at the distal end portion of the insertion unit and generating ultrasonic vibration to conduct a treatment of a region to be treated, and
driving means for driving the treatment portion,
the ultrasonic treatment device performing a treatment to the region to be treated by ultrasonic vibration of the treatment portion within an observation view field obtained by the endoscope, wherein
the insertion unit includes a flexible sheath having a distal end portion and a proximal end portion, the distal end portion of the sheath is coupled with the treatment portion,
an operation unit for operating the treatment portion is disposed at the proximal portion of the sheath,
the driving means comprises
an actuator provided with an actuator main body formed from an electrostrictive polymer and at least a pair of electrodes disposed so as to be spaced from each other in an insulated state from each other, where the electrostrictive polymer is driven in a stretching and shrinking manner when voltage is applied between the respective electrodes, and
power source means supplying voltage between the respective electrodes of the actuator, and
the treatment portion includes a blade which is joined to the actuator and which generates ultrasonic vibration in linkage with driving of the actuator in the stretching and shrinking manner to conduct a treatment of the site to be treated.

11. The ultrasonic treatment device according to claim 10, wherein
the treatment portion includes a cylindrical blade having a distal end portion and a proximal end portion, the blade having a suction hole communicating with an inner cavity of the blade at the distal end portion, and
the sheath includes a suction tube having a distal end portion and a proximal end portion and disposed in the sheath, the distal end portion of the tube communicating with an inner cavity of the blade, and the proximal end portion of the tube extending from the operation unit to the outside to be connected to a suction pump.

12. The ultrasonic treatment device according to claim 10, wherein
the treatment portion comprises
a cylindrical blade having a distal end portion and a proximal end portion, in which the driving means is housed,
a distal end cover member having a distal end portion and a proximal end portion, the proximal end portion of the distal end cover member being coupled to the distal end portion of the sheath,
a forceps piece rotatably coupled to the distal end cover member and supported to be openable and closable to the blade,
a forceps piece driving unit driving the forceps piece in an opening and closing manner to the blade,
an operation wire having a distal end portion and a proximal end portion, provided in the sheath so as to be movable in a center axial direction of the sheath, and operating the forceps piece driving unit, and
a handle provided on the operation unit, driving the forceps piece driving unit via the operation wire, and driving the forceps piece in an opening and closing manner to the blade.
